(19) United States
(12) Patent Application Publication
(10) Pub. No.: US 2026/0224642 A1
(43) Pub. Date: Aug. 6, 2026

(54) AKKERMANSIA MUCINIPHILA AND METHOD THEREOF FOR PREVENTING NON-ALCOHOLIC STEATOHEPATITIS

(71) Applicant: Leeuwenhoek Laboratories Co. Ltd., Taipei City (TW)

(72) Inventors: Cheng-Chih HSU, Taipei City (TW); Wei-Kai WU, Taipei City (TW); Ming-Shiang WU, Taipei City (TW); Yi-Ling LO, Taipei City (TW); Chi-Shan LI, Taipei City (TW); Chiao-Wei LIN, Taipei City (TW)

(21) Appl. No.: 19/148,003

(22) PCT Filed: Aug. 2, 2024
(86) PCT No.: PCT/CN2024/109492

§ 371 Date: Jul. 15, 2025

Related U.S. Application Data
(60) Provisional application No. 63/589,991, filed on Oct. 12, 2023.

(51) Int. Cl. A61K 35/74; A61K 35/00; A61P 1/16; A61P 3/04; C12N 1/205; C12R 1/01

(52) U.S. Cl. CPC A61K 35/74; A61P 1/16; A61P 3/04; C12N 1/205; A61K 2035/11; C12R 2001/01

(57) ABSTRACT

Please amend abstract “An Akkermansia muciniphila strain LWHK0003 is provided, and the strain prevents nonalcoholic steatohepatitis, also prevents obesity, prevents NASH induced by GAN diet, maintains blood sugar, and anti-inflammation. A method of preventing nonalcoholic steatohepatitis comprising administering to a subject in need thereof an effective amount of bacterial strain as above mentioned is also provided.”

### BACKGROUND

### Field of Invention

The present invention relates to an Akkermansia muciniphila and method thereof. More particularly, the present invention relates to an Akkermansia muciniphila and method of preventing nonalcoholic steatohepatitis thereof.

### DESCRIPTION OF RELATED ART

Non-alcoholic fatty liver disease (NAFLD) is the most common liver disease. Without proper treatment, it will deteriorate into non-alcoholic steatohepatitis (NASH), which may lead to irreversible cirrhosis and hepatocellular carcinoma. The progression of NASH is closely related to metabolic syndrome and intestinal flora imbalance caused by an unhealthy diet.

“Probiotics” are active microorganisms that coexist with a host. Appropriate intake of probiotics can have a beneficial effect on the host's health. Although traditional probiotics such as Lactobacillus and Bifidobacterium have been widely used and have good safety and human tolerance, the effects on human intestinal microorganisms are still limited, and there are great differences in the benefits and mechanisms between different strains.

Therefore, how to provide probiotics that can prevent and treat NASH, the related art really needs to be improved.

### SUMMARY

The present disclosure provides an Akkermansia muciniphila LWHK0003 deposited at DMSZ under number DSM 35051.

The present disclosure also provides a composition for preventing nonalcoholic steatohepatitis, comprising: the Akkermansia muciniphila LWHK0003 as above mentioned; and a pharmaceutical acceptable carrier.

In some embodiments, the Akkermansia muciniphila LWHK 0003 is dead strain.

In some embodiments, the dead strain was pasteurized.

The present disclosure also provides a use of bacterial strain in the manufacture of a drug or a health food for preventing nonalcoholic steatohepatitis, wherein the bacterial strain is Akkermansia muciniphila LWHK0003 as above mentioned.

In some embodiments, the bacterial strain is a live strain, a dead strain, or a combination thereof.

In some embodiments, the bacterial strain prevents nonalcoholic steatohepatitis accompanied by followings comprising obesity, high fat, high liver triglycerides, high nonalcoholic fatty liver disease activity score (NAS), high blood triglycerides, or a combination thereof.

In some embodiments, the bacterial strain prevents nonalcoholic steatohepatitis accompanied by high fat comprising body fat, visceral fat or a combination thereof.

In some embodiments, the bacterial strain prevents nonalcoholic steatohepatitis accompanied by high NAS comprising hepatocyte ballooning, liver inflammation, or a combination thereof.

In some embodiments, the drug or the health food is in a form of capsule, tablet, powder or liquid.

In some embodiments, the drug or the health food is formulated for oral delivery.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion. The disclosure can be more fully understood by reading the following detailed description of the embodiment, with reference made to the accompanying drawings as follows:

FIG. 1A shows body weights (statistical analysis using t-test) of mice at week 8 according to one embodiment of the present disclosure.

FIG. 1B shows changes in body weight (statistical analysis using t-test) of mice during 8 weeks according to one embodiment of the present disclosure.

FIG. 2A shows weight gain (statistical analysis using t-test) of mice at week 8 according to one embodiment of the present disclosure.

FIG. 2B shows changes in weight gain (statistical analysis using t-test) of mice during 8 weeks according to one embodiment of the present disclosure.

FIG. 3 shows a relative adipose tissue weight of mice according to one embodiment of the present disclosure, including subcutaneous adipose tissue (SAT), epididymal adipose tissue (EAT), perirenal adipose tissue (PRAT) and brown adipose tissue (BAT) weights (statistical analysis using t-test).

FIG. 4 shows contents (statistical analysis using t-test) of triglycerides (TG) in the liver of mice according to one embodiment of the present disclosure.

FIGS. 5A to 5C show pathological sections of mice liver tissue according to one embodiment of the present disclosure.

FIG. 6A shows steatosis scores (statistical analysis using Mann-Whitney U test) of mice liver according to one embodiment of the present disclosure.

FIG. 6B shows hepatocyte ballooning scores (statistically using Mann-Whitney U test) of mice according to one embodiment of the present disclosure.

FIG. 6C shows inflammation scores (statistical analysis using Mann-Whitney U test) of mice liver tissue according to one embodiment of the present disclosure.

FIG. 6D shows liver tissue pathological section and a non-alcoholic fatty liver activity score (NAFLD activity score, NAS) (statistics using Mann-Whitney U test) of mice according to one embodiment of the present disclosure.

FIG. 7A shows triglyceride contents in blood (t-test) of mice according to one embodiment of the present disclosure.

FIG. 7B shows blood cholesterol levels (statistical analysis using t-test) in mice according to one embodiment of the present disclosure.

FIG. 8 shows blood glucose levels (statistical analysis using t-test) of mice after eating according to one embodiment of the present disclosure.

FIG. 9A illustrates an oral glucose tolerance test (OGTT) (statistical analysis using t-test) in mice according to one embodiment of the present disclosure.

FIG. 9B illustrates area under the curves (AUC) (statistical analysis using t-test) of blood glucose in mice according to one embodiment of the present disclosure.

### DETAILED DESCRIPTION

The following disclosure provides detailed description of many different embodiments, or examples, for implementing different features of the provided subject matter. These are, of course, merely examples and are not intended to limit the invention but to illustrate it. In addition, various embodiments disclosed below may combine or substitute one embodiment with another, and may have additional embodiments in addition to those described below in a beneficial way without further description or explanation. In the following description, many specific details are set forth to provide a more thorough understanding of the present disclosure. It will be apparent, however, to those skilled in the art, that the present disclosure may be practiced without these specific details.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

The strain of Akkermansia muciniphila LWHK0003 of present disclosure has been deposited at bioresource collection and research center (BCRC) in Taiwan with the address being No. 331, Shih-Pin Road, Hsinchu, 300 Taiwan, and the deposit number is BCRC911209, and the deposit date is Nov. 21, 2023. The strain of Akkermansia muciniphila LWHK0003 of present disclosure also has been deposited at DSMZ (Deutsche Sammlung von Mikroorganismen und Zellkulturen) with the address being Inhoffenstr 7B, 38124 Braunschweig, Germany, and the deposit number is DSM 35051, and the deposit date is Jun. 14, 2024.

The Akkermansia muciniphila LWHK0003 disclosed in the present disclosure is an anaerobic bacterium isolated from the feces of healthy humans in Taiwan. The strain is oval in appearance, with a diameter of about 0.4 micrometers (μm) to 0.6 μm and a length of about 0.6 μm to 1 μm. The Akkermansia muciniphila live strain disclosed in the present disclosure was cultured in brain heart infusion broth (BHI broth) in an anaerobic environment at 37° C.

In some embodiments of the present disclosure, the strain is administered to a subject orally or parenterally. In some embodiments of the present disclosure, the strain is formulated into an oral dosage form selected from the group consisting of solution, suspension, emulsion, powder, lozenge, pill, syrup, buccal lozenge, tablet, chewing gum and capsule for administration to a subject.

In some embodiments, the pharmaceutically acceptable carrier comprises, but is not limited to water, alcohols, glycol, preserving agents, antioxidants, solvent, emulsifier, suspending agent, decomposer, binding agent, excipient, stabilizing agent, chelating agent, diluent, gelling agent, preservative, lubricant, absorption enhancers, active agents, humectants, odor absorbers, fragrances, pH adjusting agents, occlusive agents, emollients, thickeners, solubilizing agents, penetration enhancers, anti-irritants, colorants, propellants, surfactant, and other similar or applicable carriers for the present invention.

As used herein, the term “GAN diet” refers to Gubra-Amylin NASH. In the past, a high-fat diet called the ALIOS (American lifestyle-induced obesity syndrome) diet promoted experimental NASH. Hansen et al. further improved it and named it the Amylin liver NASH (AMLN) diet, which can reliably induce NASH metabolic biochemistry, liver histopathology and other manifestations in C57BL/6J mice (AMLN diet-induced NASH, AMLN DIO NASH) and ob/ob mice lacking leptin (AMLN ob/ob-NASH). However, since FDA banned the use of trans fats as food additives in 2018, the GAN diet, which is similar to the AMLN diet but does not contain trans fats, was born. Specifically, the GAN diet is diet #D09100310i (Source: Research Diets, INC) containing 40 kcal % fat, 20 kcal % fructose, and 2% cholesterol.

A number of examples are provided herein to elaborate the Akkermansia muciniphila and use of preventing nonalcoholic steatohepatitis thereof of the present disclosure. However, the examples are for demonstration purpose alone, and the instant disclosure is not limited thereto.

### EXAMPLE

### Experimental Group

C57BL/6 male mice were divided into three groups and all were given a GAN diet. In addition to the GAN diet, the positive control group (hereinafter referred to as BAA-835) was fed with 1×10⁹ CFU/200 μL (PBS containing 2.5% glycerol) of Akkermansia muciniphila (ATCC BAA-835) pasteurized bacteria per tube per day. In addition to the GAN diet, the experimental group (hereinafter referred to as LWHK03) was fed with 1×10⁹ CFU/200 μL (PBS containing 2.5% glycerol) of Akkermansia muciniphila (LWHK0003) per tube per day. In addition to the GAN diet, the control group (Con) was fed with 200 μL of PBS containing 2.5% glycerol per tube per day, the volume was the same as the positive control group and the experimental group. Body weight, food intake, and water intake were recorded weekly. An oral glucose tolerance test (OGTT) was performed at week 7, and mice were sacrificed at week 8 to collect blood, multiple fat, and liver samples.

### Example 1 Weight Gain

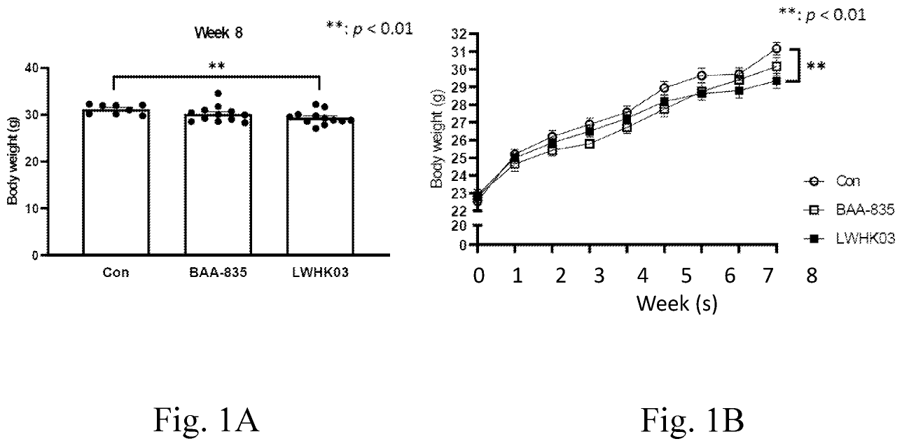

The experimental groups were as described above, and the body weights from week 0 to week 8 were recorded. The results were shown in FIGS. 1A and 1B. It was obvious that at week 8, the average body weight of the control group mice was 31.17 grams, and the LWHK03 group reduced the average body weight of GAN diet-induced nonalcoholic steatohepatitis (NASH) mice to 29.36 grams, and the effect was better than the average of 30.16 grams in BAA-835 group.

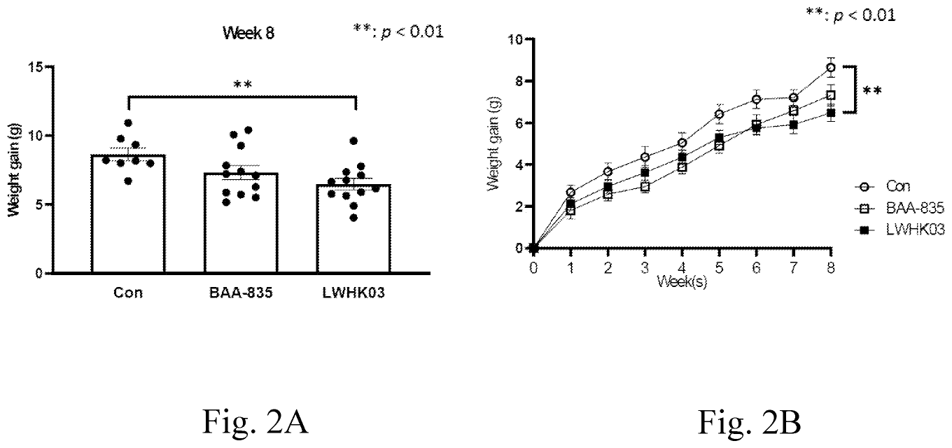

Furthermore, as shown in FIG. 2A and FIG. 2B, at week 8, the LWHK03 group inhibited the average weight gain of GAN diet-induced nonalcoholic steatohepatitis (NASH) mice by 6.48 grams, and the effect was better than that of the BAA-835 group by 7.32 grams. The average weight gain of the control group mice was 8.64 grams.

### Example 2 Fat Weight

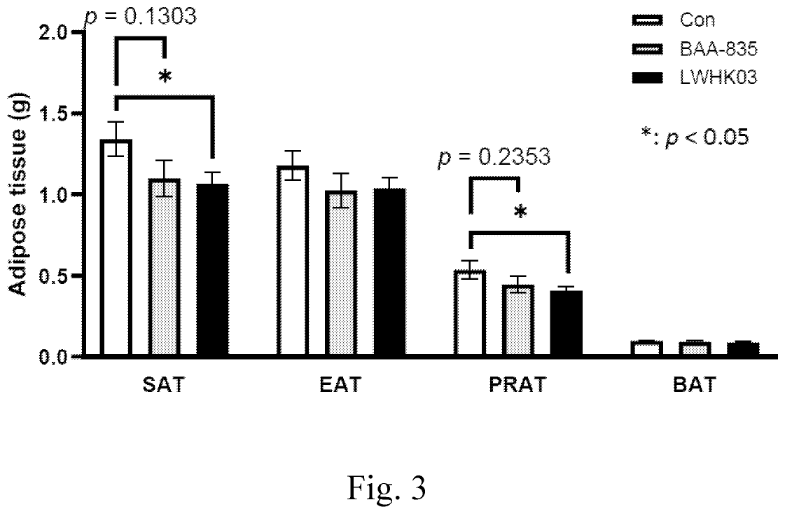

The experimental groups were as described above. Mice were sacrificed at week 8 and fat was collected from multiple sites. The results were shown in FIG. 3. At week 8, the average subcutaneous fat (SAT) weight of mice in the control group was 1.34 grams, and the LWHK03 group significantly reduced the subcutaneous fat (SAT) weight to an average of 1.07 grams, which was better than the average of 1.1 grams in the BAA-835 group. In addition, the average perirenal fat (PRAT) weight of mice in the control group was 0.54 grams, and the LWHK03 group significantly reduced the perirenal fat (PRAT) weight to an average of 0.41 grams, which was better than the average of 0.45 grams in the BAA-835 group.

### Example 3 Liver Triglyceride Content

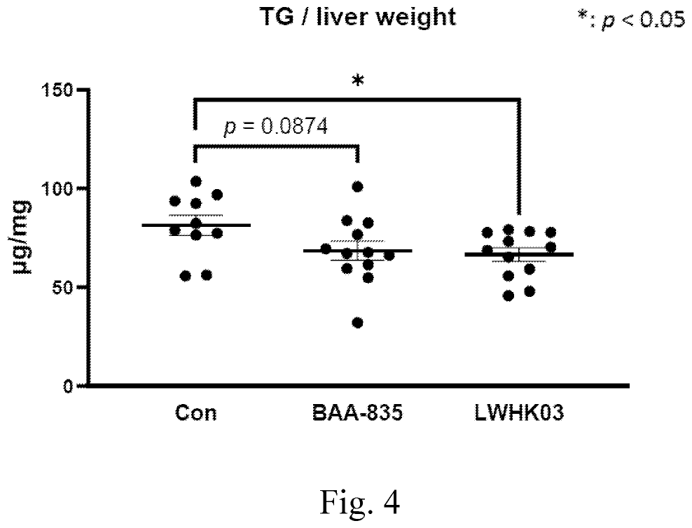

The experimental groups were as described above. Mice were sacrificed at 8 weeks and liver tissues were collected. The triglyceride content of the mouse liver was divided by the liver weight to obtain the ratio of triglyceride/liver weight. The results were shown in FIG. 4. After 8 weeks of GAN diet, the average ratio of triglyceride/liver weight in the control group was 81.39 μg/mg, and the LWHK03 group significantly reduced the average ratio of triglyceride/liver weight to 66.6 μg/mg, and the effect was better than the average of 68.57 μg/mg in the BAA-835 group.

### Example 4 Liver Pathological Sections and Liver Pathological Scores

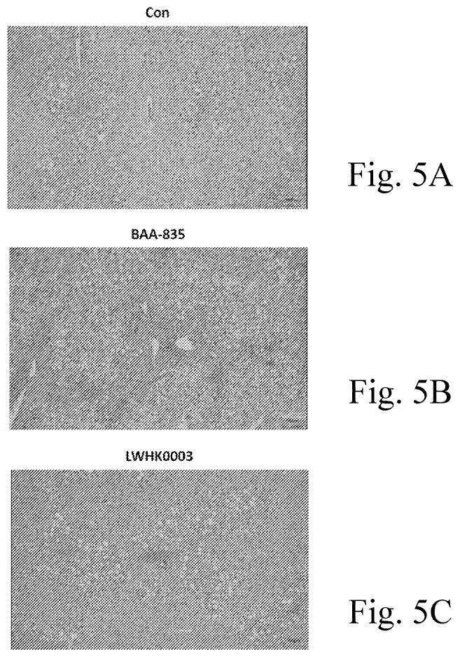

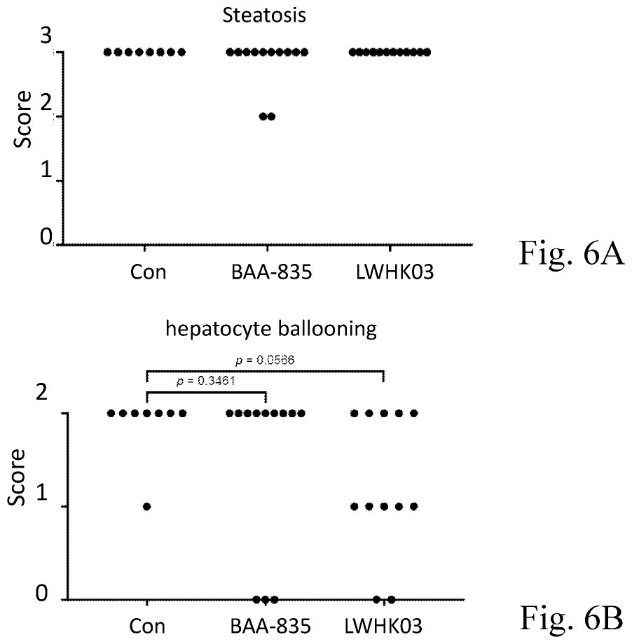

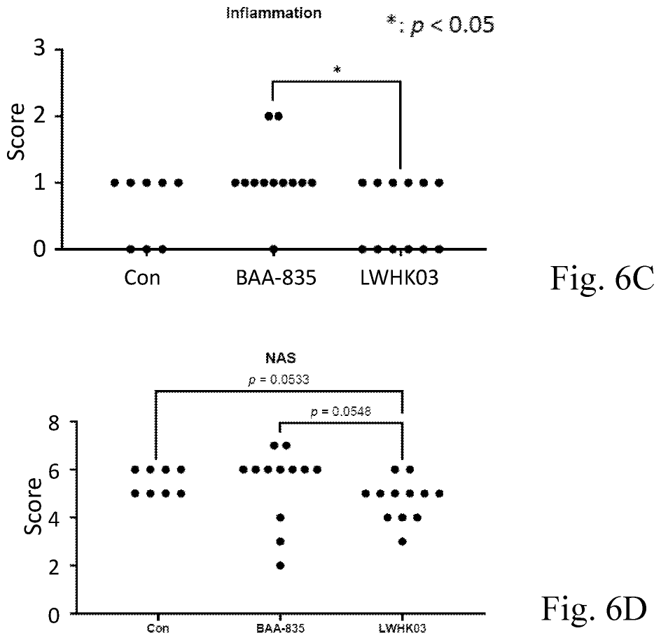

The experimental groups were as described above. Mice were sacrificed and liver tissues were collected at week 8. The sections were stained (FIGS. 5A to 5C) and interpreted according to Kleiner et al. published in 2005. The pathologist calculated the scores according to the severity of pathological features such as liver fatty degeneration, hepatocyte ballooning and liver tissue inflammation (FIGS. 6A to 6C). The scores of various NAS indicators and the total score (FIG. 6D) were calculated according to Kleiner et al. published in 2005. The full score is 8 points. If the total score is ≥5 points, NASH is diagnosed. The higher the score is, the severe the NASH condition is.

The results showed that after 8 weeks of GAN diet, there was no significant difference in steatosis among the groups (FIG. 6A). In the index of hepatocyte ballooning (FIG. 6B), among the 8 mice in the control group, score 1 had 1 mouse, and score 2 had 7 mice. Among the 12 mice in the LWHK03 group, score 0 had 2 mice, score 1 had 5 mice, and score 2 had 5 mice. Compared with the control group, the degree of hepatocyte ballooning in the LWHK03 group was significantly lower than that of the 12 mice in the BAA-835 group with score 0 had 3 mice and score 2 had 9 mice. In the indicators of inflammatory response (FIG. 6C), among the 8 mice in the control group, score 0 had 3 mice and score 1 had 5 mice. Among the 12 mice in the LWHK03 group, score 0 had 6 mice and score 1 had 6 mice. Compared with the control group, the degree of inflammatory response in the LWHK03 group was significantly lower than that of the 12 mice in the BAA-835 group with 0 score having 1mouse, 1 score having 9 mice, and score 2 having 2 mice. In the indicators of NAS (FIG. 6D), among the 8 mice in the control group, a total score of 5 had 4 mice and the total score of 6 had 4 mice. Among 12 mice in the LWHK03 group, the total score of 3 had 1 mouse, the total score of 4 had 3 mice, the total score of 5 had 6 mice, and the total score of 6 had 2 mice. Compared with the control group, the NAS index in the LWHK03 group was significantly lower than that of the 12 mice in the BAA-835 group with the total score of 2 had 1 mouse, the total score of 3 had 1 mouse, the total score of 4 had 1 mouse, the total score of 6 had 7 mice, and the total score of 7 had 2 mice.

### Example 5 Blood Lipid Analysis

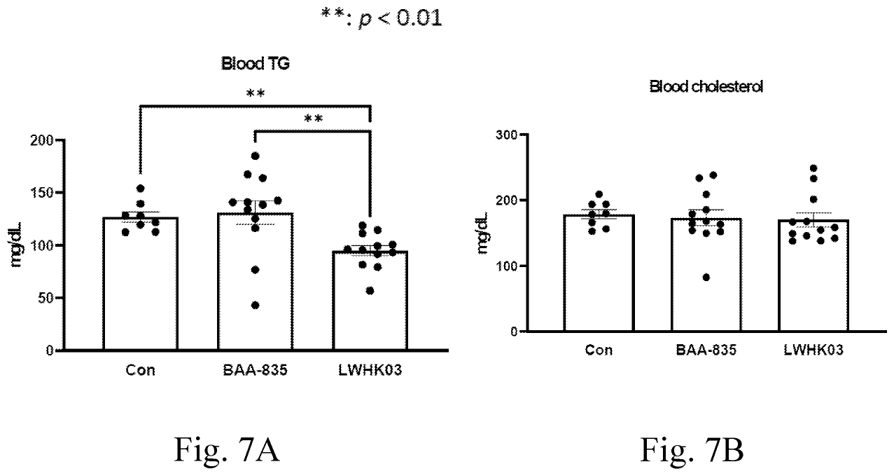

The experimental groups were as described above. Mice were sacrificed and blood was collected at week 8. The results were shown in FIG. 7A. After 8 weeks of GAN diet, the average blood triglyceride level in the control group was 126.98 mg/dL. The LWHK03 group significantly reduced blood triglyceride to an average of 94.92 mg/dL, and the effect was significantly better than the average of 131.17 mg/dL in the BAA-835 group. There was no significant difference in blood cholesterol among the three groups (FIG. 7B).

### Example 6 Blood Glucose Analysis

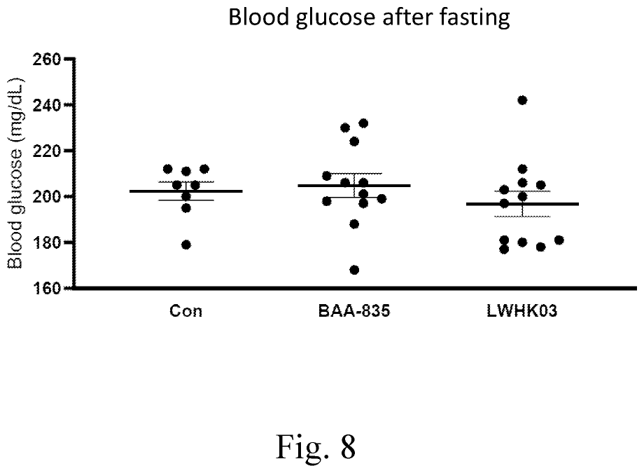

The experimental groups were as described above. At week 7 of the experiment, blood was collected after fasting for 6 hours. The results were shown in FIG. 8, there was no significant difference in blood glucose levels among the three groups after fasting.

### Example 7 Oral Glucose Tolerance Test (OGTT)

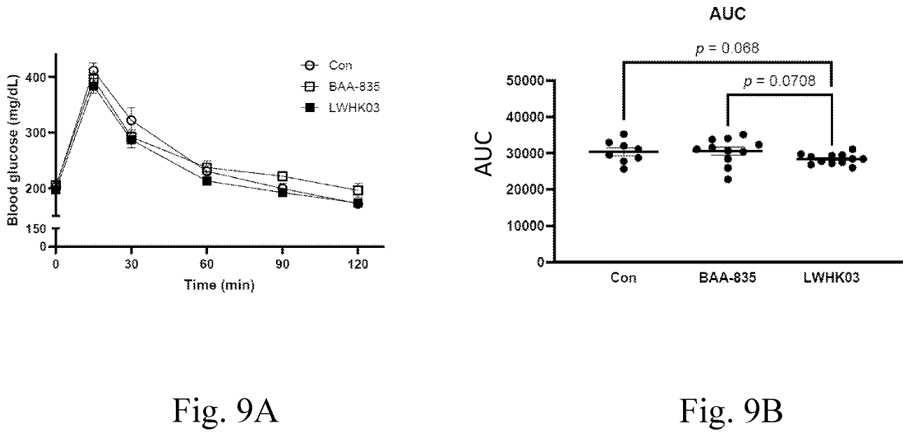

The experimental groups were as described above, and OGTT was performed at week 7. The results were shown in FIG. 9A, there was no significant difference in blood glucose levels among the three groups at different time points. Further analysis of the area under the blood glucose curve (AUC) showed (FIG. 9B) that the control group had an average AUC of 28,418, while the LWHK03 group (average AUC of 30,390) had a better effect on improving blood glucose control than BAA-835 (average AUC of 30,577).

The Akkermansia muciniphila LWHK0003 strain disclosed herein has the effects of preventing obesity, reducing weight gain, reducing fat accumulation (such as subcutaneous fat, epididymal adipose tissue and perirenal adipose tissue), reducing liver triglyceride content, preventing NASH induced by GAN diet (such as reducing hepatocyte ballooning and reducing liver inflammatory response), reducing blood triglycerides, and maintaining blood sugar stability under GAN diet induction. Furthermore, compared with the ATCC BAA-835pasteurized bacteria group, the Akkermansia muciniphila LWHK0003 strain (pasteurized bacteria) disclosed herein has better aforementioned effects.

While the disclosure has been described by way of example(s) and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

1. An Akkermansia muciniphila LWHK0003 deposited at DMSZ under number DSM 35051.

2. A composition for preventing nonalcoholic steatohepatitis, comprising:
the Akkermansia muciniphila LWHK0003 as claimed in claim 1; and
a pharmaceutical acceptable carrier.

3. The composition of clam 2, wherein the Akkermansia muciniphila LWHK0003 is dead strain.

4. The composition of clam 3, wherein the dead strain was pasteurized.

5. A method of preventing nonalcoholic steatohepatitis comprising administering to a subject in need thereof an effective amount of bacterial strain, wherein the bacterial strain is Akkermansia muciniphila LWHK0003 as claimed in claim 1.

6. The method of claim 5, wherein the bacterial strain is a live strain, a dead strain, or a combination thereof.

7. The method of claim 5, wherein the bacterial strain prevents nonalcoholic steatohepatitis accompanied by followings comprising obesity, high fat, high liver triglycerides, high nonalcoholic fatty liver disease activity score (NAS), high blood triglycerides, or a combination thereof.

8. The method of claim 7, wherein the bacterial strain prevents nonalcoholic steatohepatitis accompanied by high fat comprising body fat, visceral fat or a combination thereof.

9. The method of claim 7, wherein the bacterial strain prevents nonalcoholic steatohepatitis accompanied by high NAS comprising hepatocyte ballooning, liver inflammation, or a combination thereof.

10. The method of claim 5, wherein the bacteria strain is in a form of capsule, tablet, powder or liquid.

11. The method of claim 5, wherein the bacteria strain is formulated for oral delivery.

12. The method of claim 5, wherein the bacteria strain is administered as a drug or a health supplement.

13. The method of claim 12, wherein the drug comprises a pharmaceutically acceptable carrier.

14. The method of claim 13, wherein the pharmaceutically acceptable carrier is selected from the group consisting of micro-crystalline cellulose, trehalose, maltodextrin, rice flour, magnesium stearate, inositol, dextrose, sucrose, and a combination thereof.

15. The method of claim 12, wherein the health supplement comprises a food acceptable carrier.
